(19) United States
(12) Patent Application Publication
(10) Pub. No.: US 2024/0125723 A1
(43) Pub. Date: Apr. 18, 2024

(54) TEST SYSTEM AND METHOD FOR MEASURING EFFICIENCY OF UNDERGROUND COAL GASIFICATION

(71) Applicant: Anhui University Of Science & Technology, Huainan (CN)

(72) Inventors: Qinghe ZHANG, Huainan (CN); Fawang YANG, Huainan (CN); Liang YUAN, Huainan (CN); Pingsong ZHANG, Huainan (CN); Zhiwei LIANG, Huainan (CN); Xiaorui WANG, Huainan (CN)

(73) Assignee: Anhui University Of Science & Technology, Huainan (CN)

(21) Appl. No.: 18/507,254

(22) Filed: Nov. 13, 2023

Related U.S. Application Data
(63) Continuation of application No. PCT/CN2023/095026, filed on May 18, 2023.

(30) Foreign Application Priority Data
Oct. 17, 2022 (CN) 202211267197.6

(51) Int. Cl. G01N 25/32; G01N 33/24

(52) U.S. Cl. CPC G01N 25/32; G01N 33/24

(57) ABSTRACT

The present disclosure provides a test system and a method for measuring efficiency of underground coal gasification, and relates to the technical field of underground coal gasification, comprising a gasification agent supply system, a gasifier, an outlet and recycling system and a monitoring system, wherein the gasification agent is mainly injected into the gasifier and then chemically reacts with the coal by igniting the gasifier at a high temperature to generate gas, while after the gas passes through the outlet and recycling system, the composition and content are measured, and the monitoring system monitors an operating state of the gasifier, injection of the gasification agent and production of gas, temperature change in the gasification channel and ignition temperature and pressure gauge data.

### CROSS-REFERENCE TO RELATED APPLICATIONS

The present application claims priority to Chinese Patent Application No. 202211267197.6, entitled “TEST SYSTEM AND METHOD FOR MEASURING EFFICIENCY OF UNDERGROUND COAL GASIFICATION”, filed on Oct. 17, 2022 before China National Intellectual Property Administration, the entire contents of which are incorporated herein by reference.

### TECHNICAL FIELD

The present disclosure relates to the field of underground coal gasification technology, and in particular to a test system and method for measuring efficiency of underground coal gasification.

### BACKGROUND

The statements in this section merely provide background information related to the present disclosure and may not necessarily constitute prior art.

With the arrival of the scientific and technological revolution, the world energy is forming a new wave of multi-energy revolution, such as coal clean revolution, unconventional oil and gas revolution, new energy revolution and intelligent revolution, and the technology of human using energy is developing from high-carbon to low-carbon, non-carbon. “Underground coal gasification” will likely become the new force of this wave.

Underground coal gasification (UCG) is the process of controlled combustion of underground coal to produce combustible gases by thermal and chemical action on the coal. Gaseous fuels can be used for power generation, hydrogen production, or feedstock synthesis (e.g. synthetic natural gas and synthetic oil). The underground coal gasification has a high energy density and has a strong correlation with the petroleum and petrochemical industry. The industrial chain of “underground coal gasification-hydrogen used in petrochemical refineries-CO 2 to improve the recovery of crude oil and burial” is used to create a demonstration zone of net zero emission of petroleum and petrochemical cycle economy. It can not only make clean use of a large number of idle coal resources in deep strata, relieve the shortage of natural gas supply, but also effectively solve the environmental problems caused by CO2 emissions from coal combustion, and can more effectively reserve resources and technologies for the “hydrogen economy” era.

In the case that natural gas resources can not fully meet the rapidly increasing market demand and new energy has not yet replaced oil and gas to achieve low-cost scale supply, the key to solve energy environment problems is to strengthen the efficient clean use of coal. In the prior art, underground coal gasification model test, field test and numerical simulation have accumulated a large amount of data on the chemical reactions and parameters occurring in the gasification channel, and made an important contribution to the guidance of mining work. However, there are few tests on the gasification efficiency of coal and the temperature change in the gasification channel in the process of underground gasification coal seam, and it is impossible to determine what gasification conditions will be conducive to the production of more useful gas.

### SUMMARY

In order to solve the above-mentioned problems, the present disclosure proposes a test system and method for measuring efficiency of underground coal gasification, wherein the composition and content of a gas can be obtained by changing different gas conditions and monitoring, and the gasification efficiency can be verified by the composition and content of the gas to determine the gasification conditions and improve the gasification efficiency.

To achieve the above-mentioned purpose, the present disclosure adopts the following technical solution:
a test system for measuring efficiency of underground coal gasification, comprising a gasifier to which a gasification agent supply system, an outlet and recycling system and a monitoring system are connected, the gasification agent supply system comprising a compressor and a pressure vessel; the compressor being connected to the pressure vessel for compressing air into the pressure vessel; the pressure vessel being provided with a pipeline; a pressure gauge and a servo valve being provided on the pipeline;
the outlet and recycling system being composed of the pipeline, an exhaust ventilator and a combustion chamber; the exhaust ventilator being connected to the pipeline; the monitoring system including a PLC controller for individual measurement and control device.

The technical solution adopted by the present disclosure to achieve the above-mentioned purpose thereof is further described in the following content:
a test method for measuring efficiency of underground coal gasification comprising:
placing the obtained coal in the gasifier, injecting the gasification agent into the gasifier through the gasification agent supply system, performing ignition using an ignition head and a small burner, inserting an ignition portion through an inlet hole of the gasifier, it indicating successful ignition if a temperature is displayed on a thermocouple, and recording a temperature value and test time.

Further, the gasification efficiency at different temperatures can be obtained by changing the different gasification temperatures and then monitoring the content and composition of the gasification products while keeping the other gasification conditions constant.

Advantageous effects of the present disclosure compared with prior art are provided as follows.

The present disclosure will simulate underground coal gasification test by changing different gasification conditions, monitor the products and contents of gasification under different gasification conditions by a monitoring system, and obtain the gasification efficiency under different conditions through calculation, so as to determine which condition has the highest gasification efficiency, and then apply same in the field at a later stage to improve the resource utilization rate, thus changing physical coal mining into chemical gas mining, effectively alleviating the contradiction between “rich coal” and “gas shortage”, and reserving resources and providing technology for the arrival of the “hydrogen economy” era.

### BRIEF DESCRIPTION OF DRAWINGS

In order to explain the technical solutions in the embodiments of the present disclosure or in prior art more clearly, the following contents will briefly introduce the drawings which need to be used in the embodiments. It would be obvious that the drawings in the following description are only embodiments of the present disclosure, and it is possible for a person skilled in the art to obtain other drawings according to these drawings provided without involving any inventive effort.

FIG. 1 is a schematic diagram of principle of an underground coal gasification process according to an embodiment of the present disclosure;

FIG. 2 is a schematic diagram of a test system of underground coal gasification according to an embodiment of the present disclosure.

### REFERENCE NUMBERS

1. gas injection well; 2. back injection point; 3. gasifier; 4. ignition site; 5. production well; 6. gasification agent supply system; 7. export and recycling system; 8. monitoring system; 9. compressor; 10. pressure bottle; 11. pressure gauge; 12. conduit; 13. servo valve; 14. pressure sensor; 15. mixing station; 16. oxygen pressure bottle; 17. thermocouple; 18. exhaust ventilator; 19. combustion chamber; 20. PLC controller; 21. control room; 22. gas monitor; 23. gasifier lid.

### DETAILED DESCRIPTION OF THE EMBODIMENTS

The present disclosure is further illustrated in combination with the figures and the embodiments.

It should be noted that the following detailed description is exemplary and is intended to provide further explanation of the disclosure. Unless otherwise indicated, all technical and scientific terms used herein have the same meaning as commonly understood by one of ordinary skill in the art to which the present disclosure belongs.

It is noted that the terminology used herein is for the purpose of describing particular embodiments only and is not intended to be limiting of exemplary embodiments in accordance with the present disclosure. As used herein, the singular forms are intended to include the plural forms as well, unless the context clearly indicates otherwise, and it is to be understood that the term “comprise” and/or “include”, when used in this specification, specify the presence of features, steps, operations, elements, components, and/or combinations thereof.

### Embodiment 1

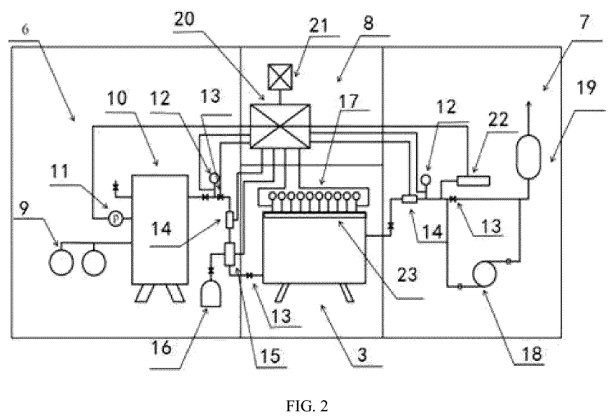

An embodiment of the present disclosure provides a test system for measuring efficiency of underground coal gasification, as shown in FIG. 2, comprising a gasifier 3 to which a gasification agent supply system 6, an outlet and recycling system 7 and a monitoring system 8 are connected, the gasification agent supply system 6 comprising a compressor 9 and a pressure vessel; the compressor 9 is connected to the pressure vessel for compressing air into the pressure vessel; the pressure vessel is provided with a pipeline 12; a pressure gauge 11 and a servo valve 13 are provided on the pipeline 12.

The outlet and recycling system 7 is composed of the pipeline 12, an exhaust ventilator 18 and a combustion chamber 19; the exhaust ventilator 18 is connected to the pipeline 12; the monitoring system 8 includes a PLC controller 20 for individual measurement and control device. The pressure vessel is a pressure bottle 10. The monitoring system 8 is used, among other things, to measure the composition and content of the gas and to control device. At the same time the pipe 12 is provided with a pressure sensor 14.

There is a test model in the gasification agent supply system 6. The gasification agent supply system 6 includes a test model such as a compressor 9, a pressure vessel, a mixing station 15 and an oxygen pressure bottle 16. The compressor 9 is used for compressing air to a pressure vessel; the amount of air supplied to the mixing station 15 is controlled by the servo valve 13, while it is also possible to supply oxygen from the oxygen pressure bottle 16 to the mixing station 15.

Further, the gasifier 3 has a rectangular shape; the gasifier 3 consists of a front portion and a vessel jacket. The gasifier 3 has a length of 3000 mm and a height of 600 mm; the entire inner surface of the gasifier 3 is an insulating layer with a thickness of 200 mm, and a gasifier lid 23 is placed above the gasifier 3; the gasifier lid 23 has 10 holes for inserting probes for temperature measurement during underground coal gasification. Two holes are provided on both sides of the gasifier 3. One of the two holes is used as input for the gasification agent and the other is used to vent gas during the test. The structure of the gasifier 3 and the arrangement of the gasification channels should ensure that the gasifier 3 has good thermal characteristics to meet the requirements of gas production continuity and stability. The structure of the gasifier 3 is a hollow wall type gasifier and a pinnate hole design, which has advantages of good seepage combustion gasification conditions and gasification reaction boundary conditions and excellent thermal characteristics.

The gasifier lid 23 is provided with ten holes; the ten holes are located on the upper side of the reactor cover of the gasifier. A thermocouple 17 is mounted on the hole of the gasifier; the thermocouple 17 is used to analyse the temperature in the coal seam channel. A gas sampling probe is tubular with a diameter of 8 mm and a length of 750 mm.

The mixing station 15 in the test system is a rectangular steel vessel. Steel containers have the advantages of light weight and high strength, good designability and excellent manufacturability. A single gas or a mixture of gases is placed in the mixing station 15; the mixture, i.e. air, is supplied and mixed simultaneously with oxygen; the output of the mixing station 15 is also the output of the entire oxygen supply system of the coal underground gasification model test process.

The compressor 9 is connected to the pressure vessel via a pressure rubber hose. The compressor 9 will automatically activate or deactivate the compressor 9 depending on the pressure requirements in the pressure vessel.

A thermocouple 17 is provided on a surface of the gasification pipeline; the thermocouple 17 is used to measure temperature to obtain data on reaction zone distribution, gasification front propagation and heat loss; the thermocouple 17 is connected to a PLC controller 20 via a compensation line.

The composition and content of the gases are measured after passing through the outlet and recycling system 7, and the incompletely combusted coal passes through the recycling system into the combustion chamber 19 for full combustion. The exhaust ventilator 18 is provided with a frequency converter, and the frequency converter is arranged on an instrument panel; and the combustion chamber 19 is made of a metal plate, and a fireproof quartz glass (a sun visor) is provided on the front side; A plurality of burners with lighters are provided on both sides of the combustion chamber 19.

The monitoring system 8 is created in the context of a visualization program of a data acquisition and monitoring control system of which the task is visualization of process variables and adjustment of controllers. A gas monitor 22 is used to monitor the operation state of the gasifier 3, upload same to a control room 21, and perform analysis via a controller 20 connected thereto, and the monitoring system 8 is used for monitoring the operation state of the gasifier 3, the injection of the gasification agent and the gas generation condition, the temperature change in the gasification channel, and the data of the servo valve 13 and the pressure gauge 11, etc.

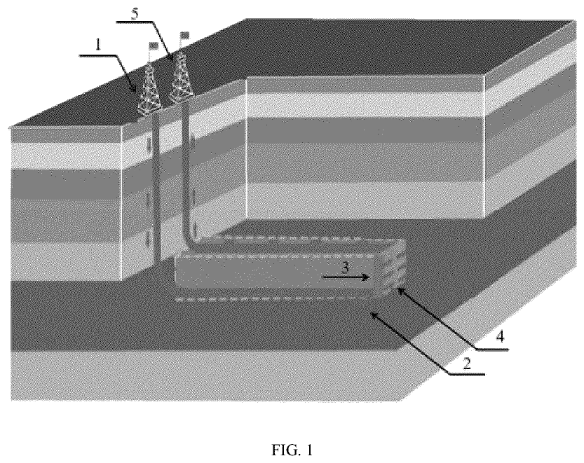

In one embodiment of the present disclosure, principle of the underground gasification process is schematically illustrated in FIG. 1. The principle of the underground coal gasification process is that a gas is introduced into a gas injection well 1, a gasification agent (air, oxygen or steam) is firstly injected, a back injection point 2 can achieve the retreat of the gasification agent, and then the gasification agent is chemically reacted with the coal by igniting the gasifier 3 at an ignition point 4 via a high temperature, and after the gas passes through the outlet and a recycling system 7, and the monitoring system 8 measures the composition and content of the gas; gaseous product is obtained in the final production zone 5; and the system can monitor the underground coal gasification in real time.

The underground coal gasification test is carried out by changing different gasification conditions, and according to the test results, the gasification efficiency under different gasification conditions is determined, so as to obtain the best gasification conditions, which lays a solid foundation for the later guidance site.

### Embodiment 2

One embodiment of the present disclosure provides a test method for measuring efficiency of underground coal gasification.

The test method includes: placing the obtained coal in the gasifier 3, injecting the gasification agent into the gasifier 3 through the gasification agent supply system 6, performing ignition using an ignition head and a small burner, inserting an ignition portion through an inlet hole of the gasifier 3, it indicating successful ignition if a temperature is displayed on a thermocouple 17, and recording a temperature value and test time.

Further, the gasification efficiency at different temperatures can be obtained by changing the different gasification temperatures and then monitoring the content and composition of the gasification products while keeping the other gasification conditions constant.

In the specific embodiment of the present disclosure, the underground coal gasification test is mainly carried out by changing different gasification conditions, and according to the test results, the gasification efficiency under different gasification conditions is determined, so as to obtain the best gasification conditions, which lays a solid foundation for the later guidance site.

Negligible is that the effect of changing the local pressure due to the removal of mass in the experimental gasification is not examined, and since the subsurface reactor is naturally formed, various anomalies may occur in the subsurface and the experiment is such that similar effects need not be considered.

In order to carry out the test, it is necessary to select a coal mine. In this case, 200 kg of coal from Zhongliangshan coal mine in China is selected, and the basic parameters are as follows: coal seam area (m²): 18000; seam bedding depth (m): 166.9; coal seam thickness (m): 3.2; overlay rock thickness (m): 166.9; overlay clay thickness (m): 87.3, coal seam inclination)(°): 0. The following are the most suitable conditions for underground coal gasification for coal sealing: the coal seam is located underground to a depth of between 100 and 600 m, the seam thickness is greater than 5 m, the ash content of the coal is less than 60%, the seam discontinuity is minimal and there is no aquifer adjacent (to avoid contamination of the drinking water supply).

The gasification agent (air, oxygen or steam) is supplied into the gasifier 3, and when other gasification conditions are unchanged, different test results are monitored by changing the difference of the injected gasification agent, and different gasification efficiencies are obtained. Air is injected first, then oxygen, then water vapor, and finally a mixture of the three.

The obtained coal is placed in the gasifier 3, the gasification agent is injected into the gasifier 3 through the gasification agent supply system 6, and ignition, which itself is performed using a special ignition head and a small burner, is inserted into the ignition portion through the inlet hole of the gasifier 3. The temperature displayed on thermocouple 17 indicates successful ignition and the temperature value and test time are recorded.

Different gasification temperatures are changed without changing other gasification conditions, wherein for example, the content and composition of the gasification products are monitored at 400° C., 600° C. and 800° C., to obtain at different temperatures.

The process of the test gasification is monitored 24 hours by the operator for safety reasons, the operator controls the input of the gasification agent and the negative pressure in the gasifier 3 at the output while controlling the temperature and concentration, replacing the damaged thermocouple 17 and replacing the filter of the analyzer.

The test process is recorded by an automatic monitoring system and all measured variables (pressure, flow and temperature) are recorded in a database and ready for further processing at the end of the test.

As an example, after the end of each set of tests, the duration of the tests and the weight of coal gasified are recorded, and the weight of coal gasified per hour and the coal gasification efficiency of the different gasification agents can be calculated to determine the optimal gasification conditions.

By changing the different gasification temperatures, monitoring the gasification products and contents, calculating the gasification efficiency at different temperatures and obtaining the optimal gasification temperature, monitoring the temperature change of the gasifier 3 by the monitoring system 8, the temperature curve in the coal can be obtained, the maximum temperature recorded corresponds to a starting position of the thermocouple 17 on the coal model, and, during the test, the combustion front gradually moves up to the end of the gasifier 3, and the maximum temperature is measured on the thermocouple 17.

The material balance is a comparison of a mass of input elements (i.e. coal and binder) and gasification agents (i.e. oxygen and air) in the charge, which will be compared to the output elements (i.e. unburned coal, ash, condensate and gas). One of the important factors determining the value index of gasification process is material balance. A non-measurable fraction of components is obtained based on a volume of synthesis gas. a weight of carbon in the synthesis gas and a proportion of gasified coal are calculated based on the balance of the amounts of carbon, hydrogen and oxygen in the synthesis gas. The remaining energy in the coal is used in heating processes, evaporation of water, underground coal gasification processes and heat loss from the surrounding environment.

The compressor 9 in the test system for coal underground gasification is connected to the pressure vessel during the test by using a pressure rubber hose.

The study of underground coal gasification is realized through the above-mentioned test system. By changing the different injection of gasification agent and different gasification temperature, the gasification efficiency under different conditions is obtained, and the best gasification conditions can be obtained. The experiment can control the test variables and eliminate the influence of irrelevant variables, which lays a solid foundation for the later field operation.

The present disclosure is described with reference to flowchart illustrations and/or block diagrams of methods, devices (systems), and computer program products according to embodiments of the present disclosure. It will be understood that each flow and/or block of the flowcharts and/or block diagrams, and combinations of flows and/or blocks in the flowcharts and/or block diagrams, can be implemented by computer program instructions. These computer program instructions may be provided to a processor of a general purpose computer, special purpose computer, embedded processor, or other programmable data processing device to produce a machine, such that the instructions, which are executed via the processor of the computer or other programmable data processing device, create means for implementing the functions specified in the flow or flows in the flowchart and/or block or blocks in the block diagram.

These computer program instructions may also be loaded onto a computer or other programmable data processing device to cause a series of operational steps to be performed on the computer or other programmable device to produce a computer implemented process such that the instructions which execute on the computer or other programmable apparatus provide steps for implementing the functions specified in the flow or flows in the flowchart and/or the block or blocks in the block diagram.

Although the particular embodiments of the present disclosure have been described above with reference to the accompanying drawings, it is not intended to limit the scope of the present disclosure. It will be apparent to those skilled in the art that various modifications and variations can be made in the present disclosure without involving any inventive effort, which still falls in the scope of the present disclosure.

What is claimed is:

1. A test system for measuring efficiency of underground coal gasification, comprising a gasifier to which a gasification agent supply system, an outlet and recycling system and a monitoring system are connected, the gasification agent supply system comprising a compressor and a pressure vessel; the compressor being connected to the pressure vessel for compressing air into the pressure vessel; the pressure vessel being provided with a pipeline; a pressure gauge and a servo valve being provided on the pipeline;
the outlet and recycling system being composed of the pipeline, an exhaust ventilator and a combustion chamber; the exhaust ventilator being connected to the pipeline; the monitoring system comprising a PLC controller for individual measurement and control device.

2. The test system for measuring efficiency of underground coal gasification according to claim 1, wherein the compressor is connected to a pressure vessel via a pressure rubber hose.

3. The test system for measuring efficiency of underground coal gasification according to claim 1, wherein composition and content of the gases are measured after passing through the outlet and recycling system, and incompletely combusted coal passes through the recycling system into the combustion chamber for full combustion.

4. The test system for measuring efficiency of underground coal gasification according to claim 1, wherein the gasifier has a rectangular shape and consists of a front portion and a vessel jacket, an inner surface of the gasifier is provided as an insulating layer, a cover plate is provided above, and holes are provided at both sides.

5. The test system for measuring efficiency of underground coal gasification according to claim 1 wherein the gasification agent supply system further comprises a mixing station and an oxygen pressure bottle, the oxygen pressure bottle supplying oxygen to the mixing station.

6. The test system for measuring efficiency of underground coal gasification according to claim 5 wherein the mixing station is a rectangular steel vessel for placing a single gas or a mixture of gases.

7. The test system for measuring efficiency of underground coal gasification according to claim 4, wherein regarding the holes provided on both sides, one is configured for input of gasification agent and the other is configured for removal of gas during the test.

8. The test system for measuring efficiency of underground coal gasification according to claim 1, wherein a thermocouple is provided on a surface of the gasification pipeline for measuring temperature to obtain data on reaction zone distribution or gasification front propagation and heat loss, the thermocouple being connected to the PLC controller via a compensation line.

9. A test method for measuring efficiency of underground coal gasification, comprising:
placing the obtained coal in the gasifier, injecting the gasification agent into the gasifier through the gasification agent supply system, performing ignition using an ignition head and a small burner, inserting an ignition portion through an inlet hole of the gasifier, it indicating successful ignition if a temperature is displayed on a thermocouple, and recording a temperature value and test time.

10. The test method for measuring efficiency of underground coal gasification according to claim 9, wherein a gasification efficiency at different temperatures can be obtained by changing different gasification temperatures and then monitoring content and composition of gasification products while keeping the other gasification conditions constant.
